(19) United States
(12) Patent Application Publication
(10) Pub. No.: US 2015/0257939 A1
(43) Pub. Date: Sep. 17, 2015

(54) TOE/FINGER BANDAGE

(71) Applicant: Joseph Ficchi, Howell, NJ (US)

(72) Inventor: Joseph Ficchi, Howell, NJ (US)

(21) Appl. No.: 14/216,695

(22) Filed: Mar. 17, 2014

(51) Int. Cl. A61F 13/06; A61F 13/10

(52) U.S. Cl. CPC A61F 13/068; A61F 13/105; A61F 2013/00153

(57) ABSTRACT

The present invention relates to a bandage that is designed for use preferably on a digit of a person's foot but that may also be used on the digit of a person's hand or other similar surfaces. In particular, the bandage comprises a body having at least a first side surface, a second side surface, a first end, and a second end. The bandage is designed to have adjustable arms at each of the first and second end so that an arm may be adjusted if it is difficult to wrap the respective arm around the anatomical digit and/or in between the spaces of the anatomical digits.

This application claims priority on U.S. Provisional Application Ser. No. 61/789,139, filed on Mar. 15, 2013, the disclosures of which are hereby incorporated by reference.

### FIELD OF THE INVENTION

The present invention redoes to a bandage tailored to adhere to a digit of a person's foot or hand. In particular, the bandage of the present invention includes a plurality of flexible arms that each may be removed from the bandage or adjusted in size to allow for a secure fit around the anatomical digit or other desired site or surface.

### BACKGROUND OF THE INVENTION

Even though marry different types of bandages have been constructed to accommodate different areas of the body, there still has not been a bandage that addresses the anatomical digits. Current bandages typically have bulky ends that are difficult to wrap around a toe/finger and to fit in the spaces between a toe/finger. Even bandages that have been designed for this purpose still present the foregoing problems. The present invention addresses and alleviates those problems by disclosing bandages that include adjustable size ends for wrapping around and. In the space between a toe/finger.

### OBJECTS OF TUB INVENTION

It is an object of the invention to provide a bandage that can easily wrap around the toe/finger.

It is another object of the invention to provide a bandage that has adjustable size ends to help in wrapping around and securing to a toe/finger.

It is still another object of the invention to provide a bandage for the toe/finger that is easy and convenient to use.

### SUMMARY OF THE INVENTION

The present invention relaxes to a bandage and a method of using the bandage that is designed for use preferably on a digit of a person's foot but that may also be used on the digit of a person's band or other similar surfaces. In particular, the bandage comprises a body having at least a first side surface, a second side surface, a first end, and a second end. The bandage is preferably constructed of a breathable material to allow the wound covered by the bandage to breathe. Materials that the bandage may be comprised of include materials that are used in the an for bandages tor allowing wounds to breathe. Accordingly, the bandage preferably has a porous surface. In addition, the bandage may also be used on the anatomical digit of a pet or other animal.

The font side of the bandage body is designed to be in contact with a wound on an anatomical digit and preferably includes gauze or other similar padding on at least a portion of its surface tor contacting the wound. The first and second end of the bandage each may comprise at least two flexible arms extending away from the bandage body. The flexible arms are designed to wrap around the edge of the digit to secure the bandage to the digit. The second side surface of the bandage is the side of the bandage that is exposed to the environment.

One of the common difficulties associated with applying a bandage to a digit is the bulky nature of the bandage arms and the difficulty wife wrapping the bandage arms around the digit in the small spaces between the digits. To ease this burden, the present invention includes at least one perforated line that may traverse the width of each flexible arm of the bandage. The perforated line functions as a tear ship that allows one or more of the flexible arms to be removed from the bandage if it is difficult to wrap that arm around the digit. Removing one or more of the arms from the bandage may create space for the other non-removed arms to be wrapped around the digit. In addition, there may be more than one perforated tear strip present on the arms. The additional tear strips may be closer to the end tip of the arm than the original tear strip to shorten the length of the arm. Rather than removing the whole arm from the bandage, a user may wish to remove only a certain portion of the arm to make it easier to wrap the remaining portion of the arm around the digit. Further, rather than the flexible arms, the ends of the bandage may have horizontally and/or diagonally situated perforations along at least a portion of its surface that may allow a user to separate the ends into two or more flexible arms for wrapping around a digit or other desired surface.

The first side of each arm of the bandage preferably has an adhesive coating to secure the bandage in place over the wound on the digit. The adhesive may preferably be of any of the types that are used in the art for bandages. In other embodiments, however, other adhesive means may be used seed as but not limited to, Velero® hook and loop fastening strips, snap fit engagements, etc.

The bandage may be packaged by any means used ha the art, such as but not limited to placing one or more peelable sterile paper coverings on the first side of the bandage to cover the adhesive and placing the bandage in a paper packaging.

### BRIEF DESCRIPTION OF THE DRAWINGS

FIG. 1 shows a view of a first embodiment of the first side surface of the bandage.

FIG. 2 shows a view of the second side surface of the bandage of FIG. 1.

FIG. 3 snows a view of a second embodiment of the first side surface of the bandage.

FIG. 4 shows a view of the second side surface of the bandage of FIG. 3.

FIG. 5 shows a view of a third embodiment of the first side surface of the bandage.

FIG. 6 shows a view of the second side surface of the bandage of FIG. 5.

FIG. 7 shows a view of a fourth embodiment of the first side surface of the bandage.

FIG. 8 shows a view of the second aide surface of the bandage of FIG. 7.

### DETAILED DESCRIPTION OF THE INVENTION

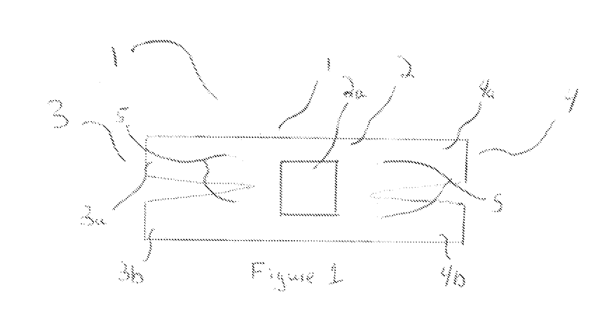

FIG. 1 shows a view of the first side surface of the bandage 1 of the present invention. Shown are the bandage body 2 and the first end 3 and second end 4 of the bandage body 2. The gauze or other similarly functioning padding 2a may be secured to a portion of the first side surface of the bandage body 2. The padding 2a is designed to be in contact with the wound on the digit. The first end and second end each comprise two flexible arms 3a and 3b and 4a and 4b extending away from the bandage body 2. The flexible arms each include a perforated line 5 traversing the width of each flexible arm. The perforated lines function as a tear strip to allow a user to tear off a flexible arm if desired. It may be desired to tear off the arm if the arm will interfere with the adhesion of the bandage to the digit or other desired surface. For example, the space may be too small to wrap the flexible arm around or the flexible arm may fit awkwardly around the digit.

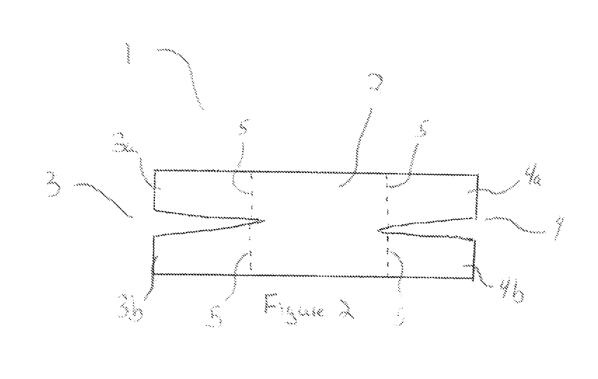

FIG. 2 is a view of the second side surface of the bandage of FIG. 1, which includes the bandage body 2 and the flexible arms 3a and 3b and 4a and 4b that comprise the first and second end 3 and 4 of the bandage, respectively. Further seen are the perforated strips 5 located on the flexible arms 3a and 3b and 4a and 4b.

It is to be noted that the ends of the bandage in other embodiments may include more than two arms. Further notably, the ends of the bandage may each contain different amounts of flexible arms. Even further notably, the top edge and bottom edge of the bandage may comprise one or more flexible arms extending away from the bandage body.

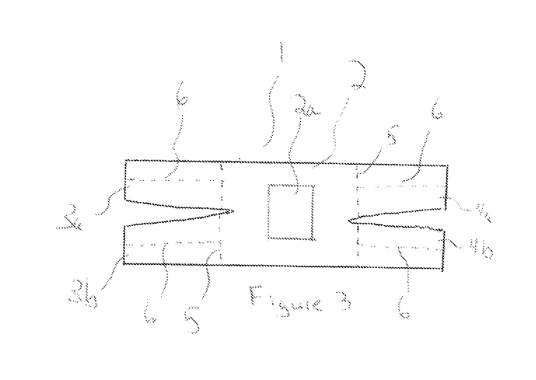

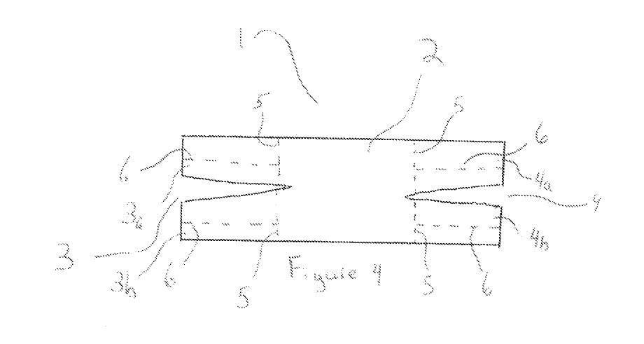

It is preferred that all of the ends, however, have at least one perforated the traversing the width of each flexible arm. One or more of the arms may also nave at least a second perforated strip extending from the first perforated strip towards the end or each flexible arm. This may allow a user to adjust the thickness of each flexible arm if desired. There may be one or more perforated strips extending from the first perforated strip to allow a user to create a variety of sixes and/or thicknesses for each flexible arm. FIGS. 3-4 shows the bandage as having a second perforated strip 6 extending away from each perforated strip 5 and towards the end of each arm.

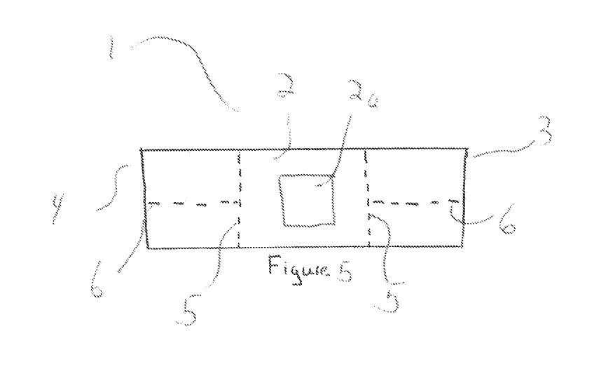

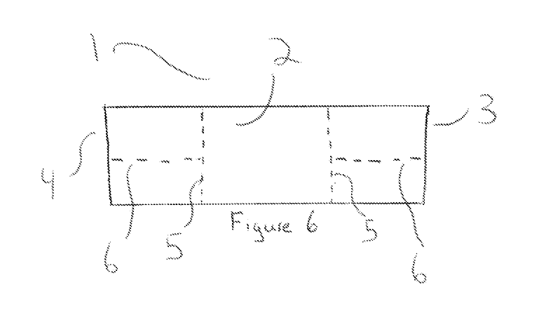

FIGS. 5-8 show the bandage as having a first perforated line 5 that traverses the width of the first and second ends 3 and 4 of the bandage and at least a second perforated line 6 that perpendicularly extends from the respective first perforated line 5 to the endpoint of each first and second end. Tearing along the strip of the second perforated line 6 allows a user to create two flexible arms at the first and second end. In addition, tearing along the strip of the first perforated line 5, after a flexible arm has been created, allows a user to remove the flexible arm from the bandage. Removing one or more flexible arms from the bandage may make it easier to wrap the bandage around the anatomical digit.

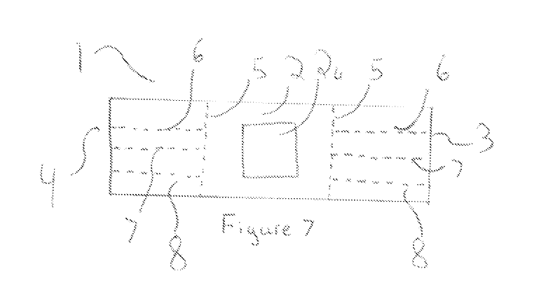

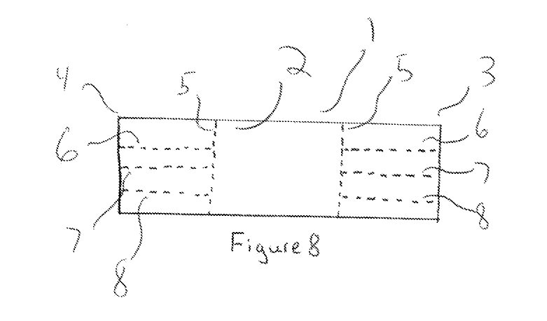

In addition to showing a second perforated line, FIGS. 7-8 show a third and fourth perforated line 7 and 8. A user may tear the second, third, and/or fourth perforated line to create more flexible arms to wrap around the anatomical digit; or a user may tear the first perforated line alone or in combination with the second, third, and/or fourth perforated line to remove all or a portion of the first and/or second ends.

Further, the side surface of the flexible arms that does not contact the anatomical digit surface may be colored to allow a user to easily identify the particular flexible arm during application of live bandage. The colors may be the same or different for each of the flexible arms or one or more of the flexible arms. Alternatively, flexible arms at the same end of the bandage may be the same color. The color of the flexible arm is preferably a color that is easily discernable from the color of the surface that the bandage is applied to. Examples of colors may be yellow, red, blue, black, green, orange, purple or white.

The flexible arms may each be labeled in addition to or rather than colored. The label may be placed at the end-tip portion of each flexible arm to identify the respective flexible arm. In this way, a user will not have to trace the flexible arm back to the body of the bandage to identity which flexible arm is being handled. The label may also be in the form of a touch label such that the label is raised and a user can feel the raised portion to determine the particular flexible arm that is being handled. Even further the second side surface of each flexible arm may also have a different texture to distinguish the flexible arms to the user of the bandage.

It is to be appreciated that the shape of the bandage is not limited to the shape depicted in the Figures. For example, the edges of the bandage may be rounded, the bandage may be a square rather than a rectangle, etc. Further and as noted herein, the flexible arms/portions of the end may be fitted with a Velcro® hook and loop fastening system, a snap fit engagement, or other means used in the art for securing the ends of a body.

What is claimed is:

1. A bandage for use on an anatomical digit of a patient comprising:
a body having a first side surface for contacting the digit a second side surface, a first end, and a second end, the first end and the second ends each comprising at least two removably attached flexible arms, wherein at least a portion of the flexible arms are removable.

2. The bandage according to claim 1 wherein the flexible arms are removably attached to the bandage by at leant one perforated strip that traverses the width of the flexible arms.

3. The bandage according to claim 1 wherein at least one of the flexible arms contains at least two perforated strips along the length of the flexible arm, the perforated strips being spaced apart along the length of the flexible arm and traversing the width of the flexible arms to allow a user to adjust the length or remove at least one of the flexible arms.

4. The bandage according to claim 2 wherein at least one perforated strip traverses the length of at least one of the flexible arms, said length traversing perforation being in communication with the width traversing perforations to allow a user to adjust the width of the flexible arm or to create an additional flexible arm.

5. The bandage according to claim 4 wherein the length traversing perforation is torn to create an additional flexible arm.

6. The bandage according to claim 5 wherein the width traversing portion of the additional flexible arm is torn to remove the additional flexible arm.

7. The bandage according to claim 2 wherein a wound care padding is located in a substantially central location on the first side surface of the bandage body, said wound care padding adapted to contact the wound or damaged portion on the skin of the patient when the bandage is applied to the patient's skin.

8. The bandage according to claim 7 wherein the first side surface of the flexible arms comprises an adhesive to adhere the bandage to the patient's skin.

9. The bandage according to claim 8 wherein the second side surface of the flexible arms are colored, the color of the flexible arms on the first end of the bandage being the same color and the color of the flexible arms on the second end of the bandage being the same color, wherein the color of the first end flexible arms is different than the color of the second end flexible arms.

10. The bandage according to claim 8 wherein the flexible arms of the bandage are wrapped around the anatomical digit as the bandage is applied to the anatomical digit, the flexible arms being wrapped around the anatomical digit and in the spaces between the anatomical digit and other anatomical digits, wherein one or more of the flexible arms may be removed from the bandage body to create space for the non-removed flexible arms to wrap around and secure the bandage to the anatomical digit.

11. The bandage according to claim 10 wherein the anatomical digit is a toe or finger of a person.

12. The bandage according to claim 8 wherein the second side of the flexible arms contains indicia identifying and distinguishing each of the flexible arms.

13. The bandage according to claim 1 wherein a plurality of perforated lines are situated diagonally on the flexible arms and/or traverse the width and/or length of the flexible arms to allow for at least a portion of the flexible arms to be removed from the bandage body or to reshape a portion of the flexible arms.

14. The method according to claim 1 wherein the flexible arms on each end of the bandage body are spaced apart.

15. A bandage tor use on an anatomical digit of a patient comprising:
a body having a first side surface for contacting the digit, a second side surface, a first end, and a second end, the first end and the second ends each comprising at least one removably and convertably attached flexible arm, wherein at least a portion of the flexible arms are removable and wherein at least a portion of the flexible arms are convertable to additional flexible arms.
